(19) United States
(12) Patent Application Publication
(10) Pub. No.: US 2015/0080905 A1
(43) Pub. Date: Mar. 19, 2015

(54) CUTTING DEVICE, HYSTERECTOMY ASSEMBLY FOR LAPAROSCOPIC HYSTERECTOMY

(71) Applicant: Mobisep B.V., Velp (NL)

(72) Inventors: Malcolm Jon Simon Begemann, Velp (NL); Wimold Pieter Steven Peters, Velp (NL); Johann Pieter Thamerus Rhemrev, Velp (NL)

(21) Appl. No.: 14/389,535

(22) PCT Filed: Apr. 2, 2013
(86) PCT No.: PCT/NL2013/050243

§ 371 Date: Sep. 30, 2014

Related U.S. Application Data
(60) Provisional application No. 61/617,809, filed on Mar. 30, 2012.

(51) Int. Cl. A61B 17/42; A61B 17/3205

(52) U.S. Cl. CPC A61B 17/42; A61B 17/3205; A61B 2017/320052
USPC 606/119; 606/167

(57) ABSTRACT

A cutting device, for example for use in a method for laparoscopic hysterectomy wherein final resection of a uterus is executed using a vaginal approach, i.e. cutting from the vaginal side inwards, instead of cutting from the abdominal side outwards of the uterus. A hysterectomy assembly for such a method is also disclosed.

### TECHNICAL FIELD

The present disclosure relates to a method and a device for removing a uterus. More particularly, the present disclosure relates to a method and a device for a novel total laparoscopic hysterectomy.

### BACKGROUND

About 70% of all hysterectomies are carried out through an abdominal incision (total abdominal hysterectomies, TAH). This requires a 2 day to 4 day hospital stay, and a 6 week to 8 week recovery time. It also produces a large, permanent abdominal scar. Only 10% of the procedures are performed with a minimally invasive approach (total laparoscopic hysterectomy, TLH). TLH has major advantages over TAH: most patients are able to return home the same or next day, with a much shorter recovery period than required for other types of hysterectomies. However, the TLH procedure is surgically more difficult to perform.

The main problems in current TLH procedures are related to difficulties in reaching and visualizing parts of the uterus with the laparoscopic tools. As can be seen in FIG. 1, the uterus is approached through small incisions on the patient's belly. In order to provide access to the desired tissue layers, the uterus needs to be moved around. A uterus mobilizer, inserted through the vagina is often used to maneuver the uterus and to create space for the laparoscopic equipment. The mobilizer does facilitate most part of dissecting the uterus, but is still limited in its ability to create sufficient room for the instruments, especially in the final phase of separating the uterus.

The location at which separation takes place is referred to as the fornix. The line of separation extends from the anterior fornix to the posterior fornix, in a 360° circular motion. The posterior side of the uterus is particularly difficult to reach. The visual feedback is poor and special caution is needed to prevent damaging surrounding structures like the bladder or intestines. These limitations lead to long procedure times. On average 15-20 minutes (up to 40 minutes) of the procedure time is spend on resolving difficulties in the actual separation of the uterus.

The disclosure is directed to a method and a device for total laparoscopic hysterectomy in which the above described problems are alleviated.

### BRIEF SUMMARY

In a first aspect, a cutting device is disclosed that includes a knife assembly that can make the initial incision from the vaginal side inwards. The cutting device is suitable for use in a method for hysterectomy wherein final resection of a uterus is executed using a vaginal approach, i.e. cutting from the vaginal side inwards, instead of cutting from the abdominal side outwards of the uterus, the cutting device. However, other applications for the cutting device are feasible as well.

In a second aspect, a manipulator device is disclosed with which the uterus may be moved in a desired direction.

In a third aspect, a hysterectomy assembly is disclosed that includes a manipulator device and a cutting device and that is configured to automatically push the uterus to a direction opposite of a cutting position where the cutting device is performing a cutting operation, thereby creating as much space and visibility of the cutting position.

In a fourth aspect a hysterectomy assembly is disclosed that is configured to execute a circular cutting action for the final resection.

In a fifth aspect, a method for laparoscopic hysterectomy is disclosed wherein final resection of a uterus is executed using a vaginal approach thereby using a hysterectomy assembly according to the present disclosure.

The vaginal approach using at least one of the disclosed devices or assemblies has major benefits. Since no other tissue layers and ligaments obstruct the separation equipment, it is easier to reach the desired location. Also, improved access and ease of navigating to the fornix decreases the procedure time considerably.

This solves the problems associated with executing the resection from the abdominal cavity, i.e.:
impaired view of the operation area;
impaired approach to the incision sites; and

Additional benefits may be:
improved manipulation by virtue of the new hysterectomy assembly with the integrated manipulator assembly and cutting assembly;
reduced operation time;
reduced risk of damaging other tissue.

The manipulator includes a manipulator element that is entered into the uterus and that may be fixated in the uterus using various fixation mechanisms. A simple fixation may be obtained by screwing the manipulator element into the uterus cavity. Once the manipulator element is in place and optionally fixated, the manipulator element may be freely rotated around a pivoting point near the entry of the uterus. The manipulation is performed manually by an operator or medical assistant during the operational procedures according to the instructions of the operating surgeon. With the hysterectomy assembly according to the invention, the manipulator element always moves the uterus away from the side of the uterus where the cutting device is active to cut the fornix.

### BRIEF DESCRIPTION OF THE DRAWINGS

FIG. 1 is a cross section of the female anatomy showing the location of the uterus with its surrounding organs;

FIG. 2 is a transparent side elevation view of an example of an embodiment of a cutting assembly;

FIG. 3 is a transparent side elevation view of the cutting housing of the cutting assembly of FIG. 2;

FIG. 4 is a transparent side elevation view of the cutting blade cover of the cutting assembly of FIG. 2;

FIG. 5 is a transparent side elevation view of the cutting blade and a lever of the cutting assembly of FIG. 2;

FIG. 6 is a side elevation view of a distal end of the cutting blade cover actuator and a distal end of the cutting blade actuator of the cutting assembly of FIG. 2

FIG. 7 shows perspective view of the example of the cutting assembly of FIG. 2 with a transparent cutting front housing part in a condition in which the cutting blade and the cutting blade cover are accommodated in the cutting device housing;

FIG. 8 shows a similar view as FIG. 7 in which both the cutting blade and the cutting blade cover protrude distally from the cutting device housing, wherein the cutting blade protrudes distally from the cutting blade cover;

FIG. 9 shows a similar view as FIG. 7 in which both the cutting blade and the cutting blade cover protrude distally from the cutting device housing, wherein the cutting blade is completely accommodated within the cutting blade cover;

FIG. 10 shows a similar view as FIG. 7 in which the cutting blade protrudes proximally from the cutting blade cover;

FIG. 11 shows a similar view as FIG. 7 in which the cutting blade is pushed against the edge of the cutting device housing;

FIGS. 12a and 12b. show an example of an embodiment via which the cutting blade cover actuator and the cutting blade actuator may be operated;

FIG. 13 shows a cross section view of a distal end of an example of an embodiment of a hysterectomy assembly in a first position;

FIG. 14 shows a similar view of the example of FIG. 13 in a second position;

FIG. 15 shows a perspective view of the example in of FIG. 13 in that position;

FIG. 16 shows a perspective view of the example of FIG. 14 in that position; and

FIG. 17a-17c show perspective views of the complete hysterectomy assembly in various positions;

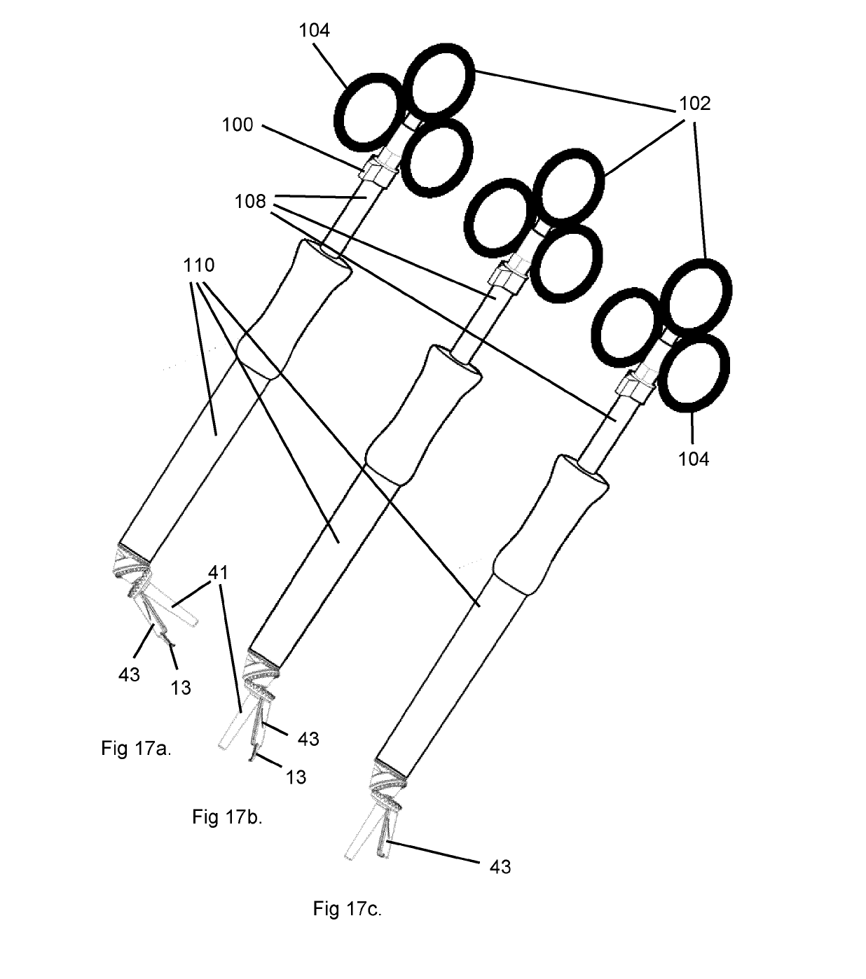

FIGS. 18a-18c show the distal parts of the hysterectomy assembly shown in FIGS. 17a-17c in more detail;

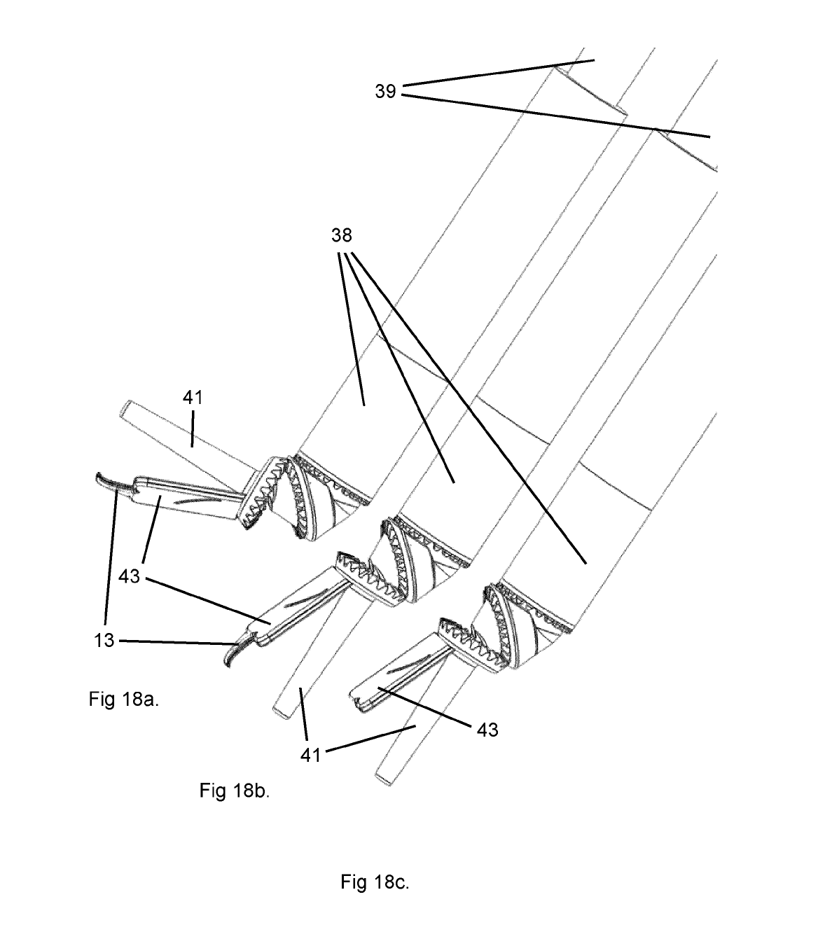

FIGS. 19a and 19b shows a cross section view of a distal end of an example of a second embodiment of a hysterectomy assembly in a first and second position; and

FIG. 20a-20e show schematically an example of an embodiment of the operation assembly for the cutting device in various positions.

### DETAILED DESCRIPTION

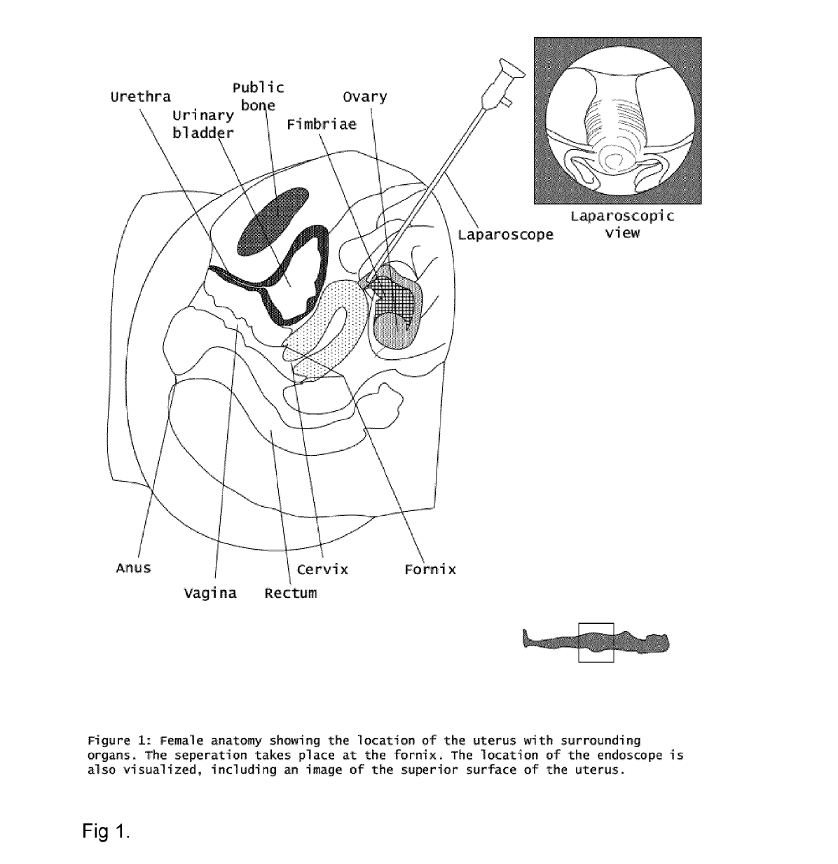

FIG. 1 shows, as stated in the background section, a cross section of the female anatomy at the location of the uterus with its surrounding organs. FIG. 1 also shows the endoscope directed to the uterus.

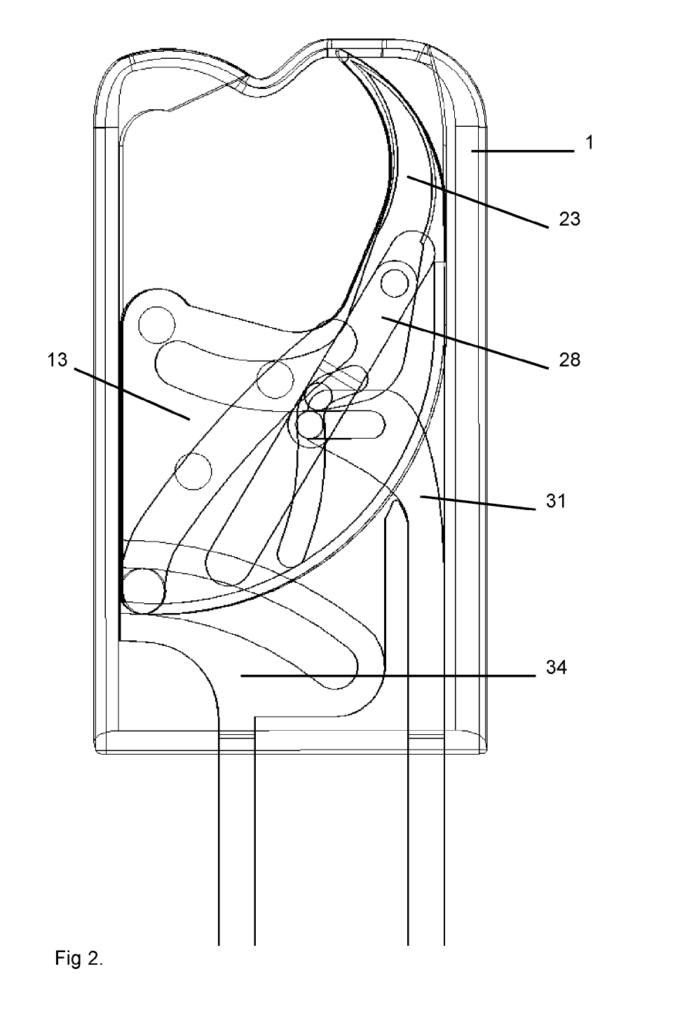

FIG. 2 is a transparent side elevation view of an example of an embodiment of a cutting assembly. The reference numbers of FIG. 2 relate to the following:
1. Cutting device housing
13. Cutting blade cover
23. Cutting blade
28. Lever
31. Cutting blade actuator;
34. Cutting blade cover actuator;

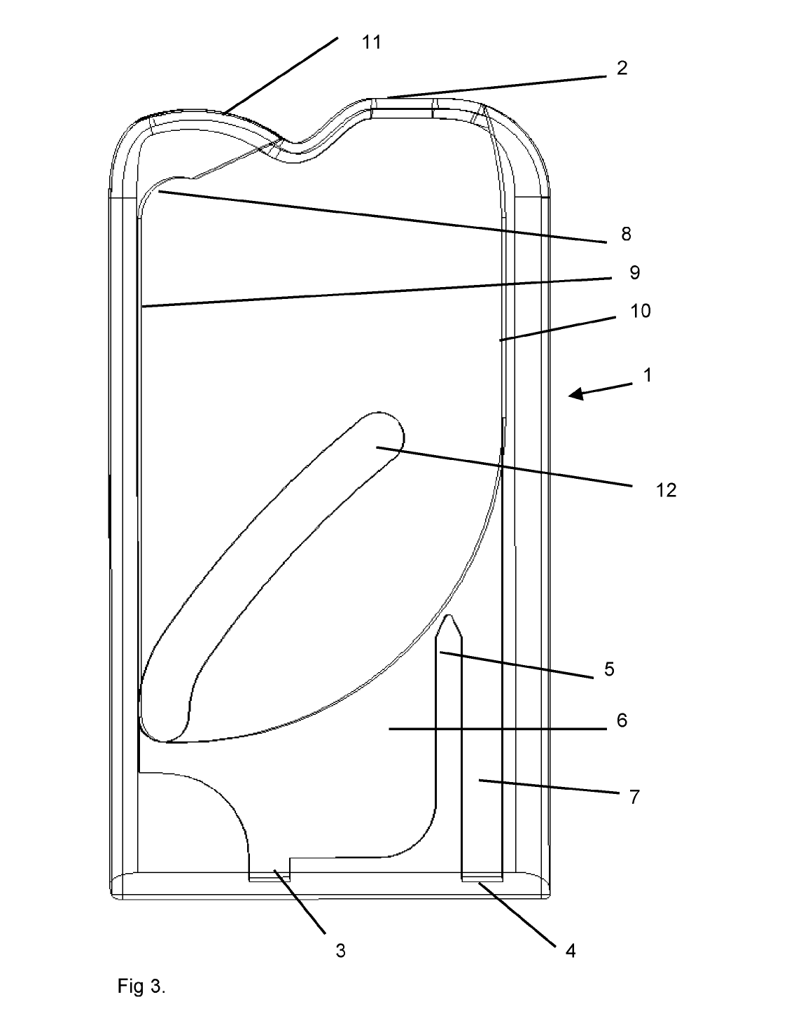

FIG. 3 is a transparent side elevation view of the cutting housing of the cutting assembly of FIG. 2. The reference numbers of FIG. 3 relate to the following:
1. Cutting device housing configured to accommodate the cutting blade cover, the cutting blade, the cutting blade cover actuator and the cutting blade actuator
2. Opening in the cutting device housing 1 to allow protrusion of the cutting blade cover 13 and the cutting blade 23.
3. Opening in the cutting device housing 1 to allow passage of the cutting blade cover actuator 34.
4. Opening in the cutting device housing 1 to allow passage of the cutting blade actuator 31.
5. Partition wall forming one side of the guiding groove 6 of the cutting blade cover actuator 34 and one side of the guiding groove 7 for the cutting blade actuator 31.
6. Guiding groove for cutting blade cover actuator 34.
7. Guiding groove for cutting blade actuator 31.
8. Rounded corner serving as a hinge for the cutting blade cover 13.
9, 10. Sides of the space in the cutting device housing 1 that serve as a guiding groove for the cutting blade cover 13.
11. Area of the cutting device housing 1 toward which the cutting blade 23 cuts.
12. Guiding groove for the notch 18 of the cutting blade cover 13.

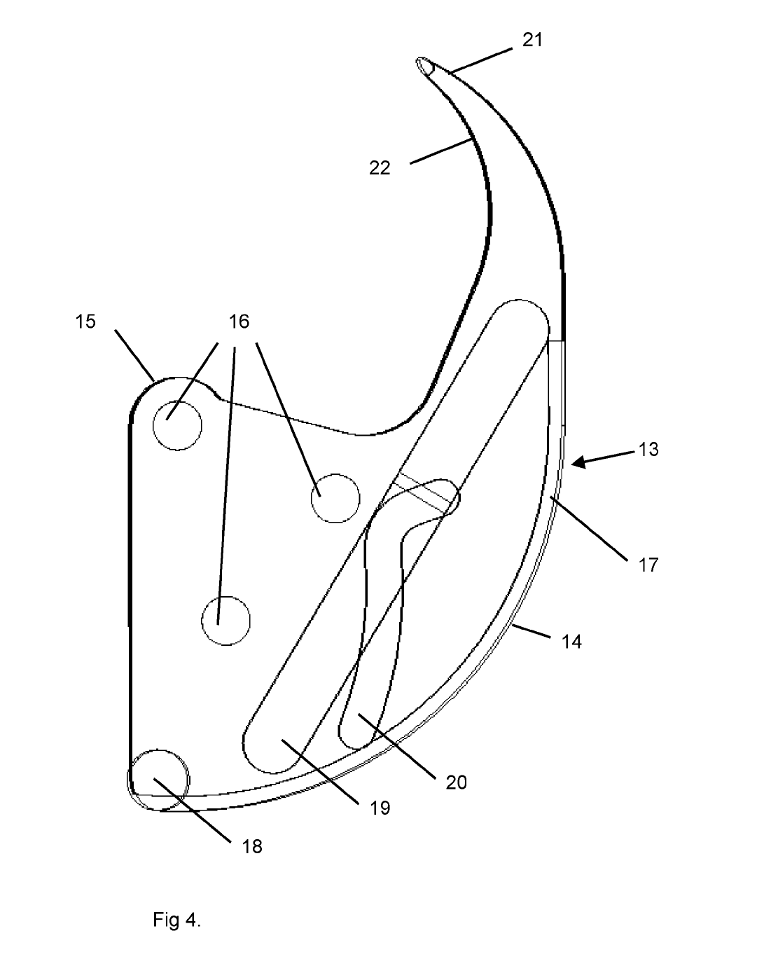

FIG. 4 is a transparent side elevation view of the cutting blade cover 13 of the cutting assembly of FIG. 2. The reference numbers of FIG. 4 relate to the following:
13. Cutting blade cover having a front part and a rear part between which the cutting blade 23 is accommodated
14. Rounded outer edge of the cutting blade cover 13.
15. Rounded inner edge of the cutting blade cover 13.
16. Elevated bulges reducing friction of the sliding movement of the cutting blade cover 13 within the cutting device housing 1. Additionally, the elevated bulges create a distance between the cutting blade cover 13 and the cutting device housing 1 to form a space in which the cutting blade actuator 31 and cutting blade cover actuator 34 extend.
17. Elevated ridge reducing friction of the sliding movement of the cutting blade cover 13 within the cutting device housing 1.
18. Notch that moves within guiding groove 12 of the cutting device housing 1 and together with rounded outer and inner edge 14, 15 limits the freedom of movement of the cutting blade cover 13 within the cutting device housing 1 to one path and related angle. Notch 18 is also engaged by the cutting blade cover actuator 34.
19. Guiding groove in a front part of the cutting blade cover 13 for co-operation with a notch 30 of the lever 28 of the cutting blade 23.
20. Guiding groove in the rear part of the cutting blade cover 13 for co-operation with one of the notches 27 of the blade 23.
21. Opening in the cutting blade cover 13 allowing protrusion of the cutting blade 23 for the stabbing function
22. Opening in the cutting blade cover 13 allowing for protrusion of the cutting blade 23 for the cutting function

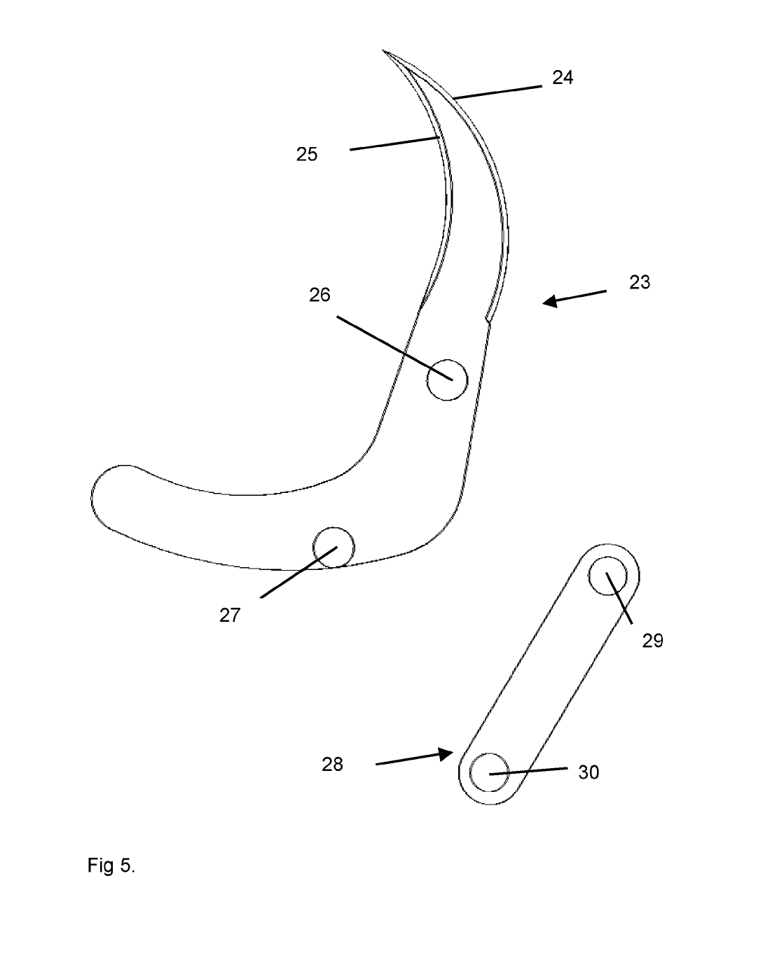

FIG. 5 is a transparent side elevation view of the cutting blade 23 and a lever 28 of the cutting assembly of FIG. 2. The reference numbers of FIG. 5 relate to the following:
23. Cutting blade.
24. Sharp edge of the cutting blade for the stabbing function.
25. Sharp edge of the cutting blade 23 for the cutting function.
26. Notch that fits in the lever 28.
27. Notch that moves within the guiding groove 20 for the cutting blade 23.
28. Lever.
29. Hole in the lever 28 for the notch 26 of the cutting blade 23
30. Notch that moves in the guiding groove 20 of the cutting blade cover 13 and is engaged by the cutting blade actuator 31.

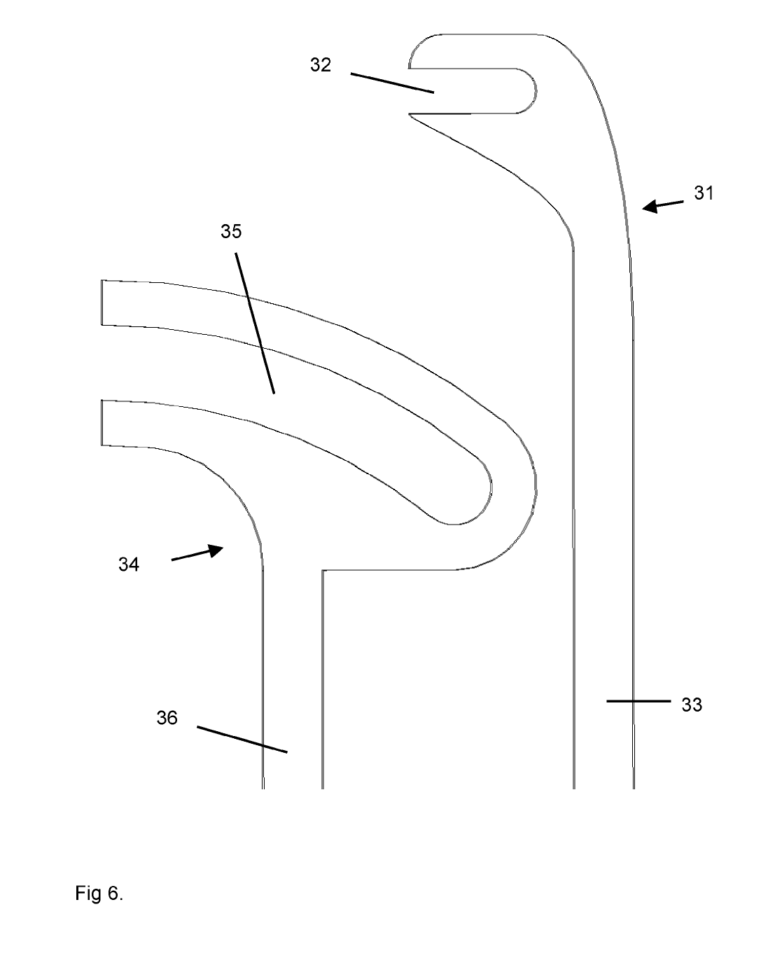

FIG. 6 is a side elevation view of a distal end of the cutting blade cover actuator 34 and a distal end of the cutting blade actuator 31 of the cutting assembly of FIG. 2. The reference numbers of FIG. 6 relate to the following:
31. Cutting blade actuator.
32. Slot for notch of the lever 28.
33. Handle of the cutting blade actuator 31 that slides in the blade handle guiding groove 7 of the cutting device housing 1.
34. Cutting blade cover actuator.
35. Slot for the notch 18 of the cutting blade cover 13.
36. Handle of the cutting blade cover actuator 34 that slides in the cover handle guiding groove 6 of the cutting device housing 1.

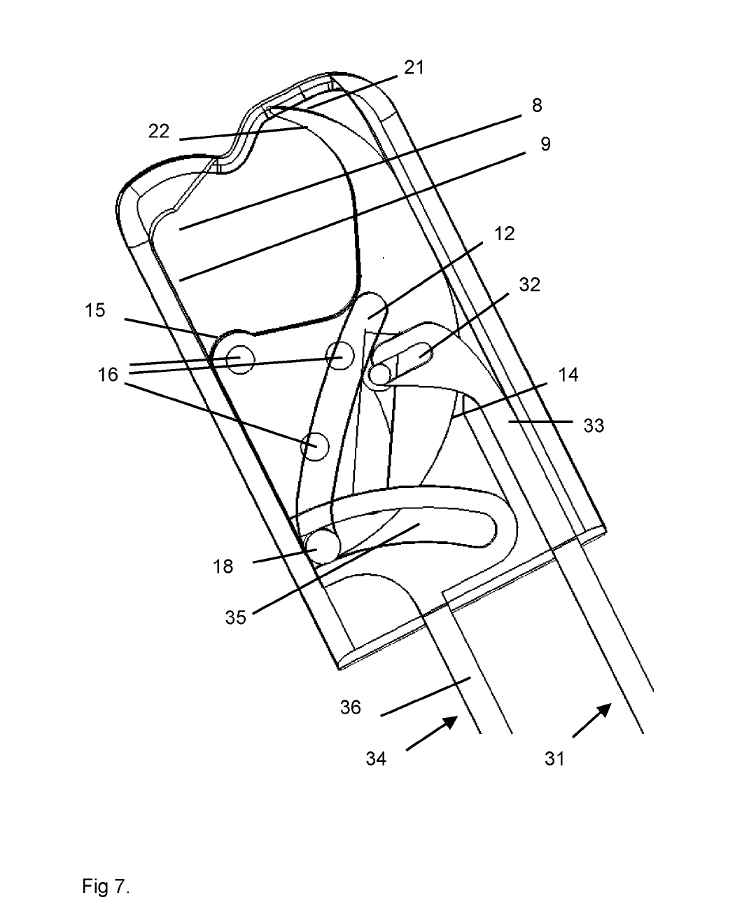

FIG. 7 shows perspective view of the example of the cutting assembly of FIG. 2 with a transparent cutting front housing part in a condition in which the cutting blade 23 and the cutting blade cover 13 are accommodated in the cutting device housing 1;

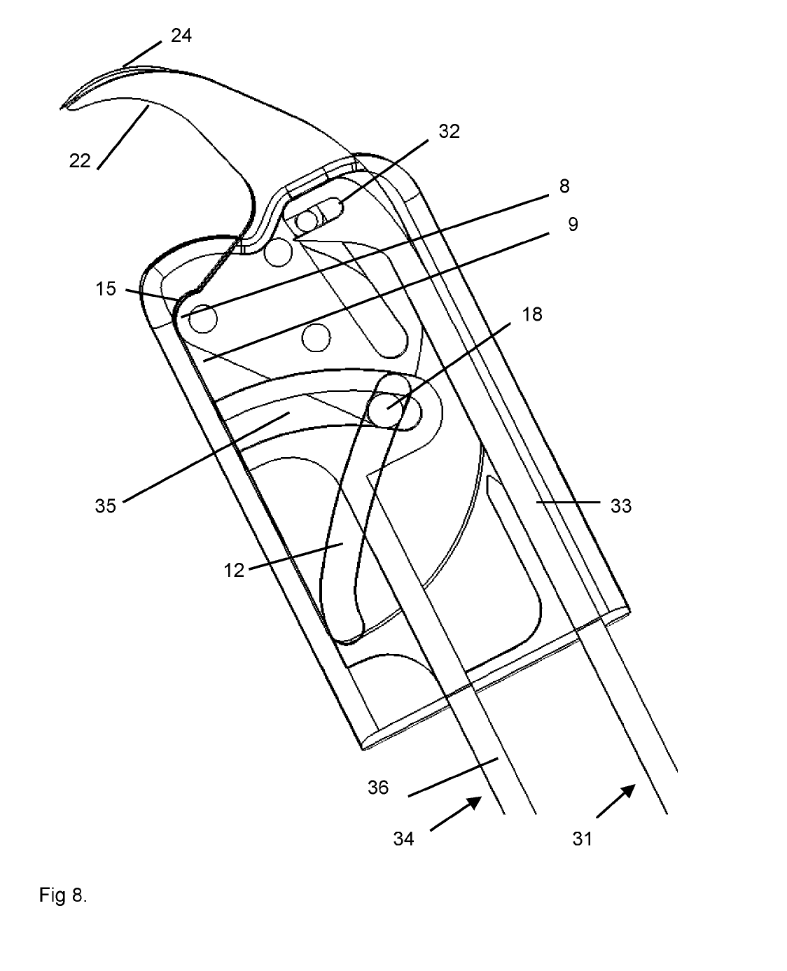

In FIG. 8 the cutting blade 23 and the cutting blade cover 13 both protrude distally from the cutting device housing 1 and wherein the cutting blade 23 protrudes distally from the cutting blade cover 13.

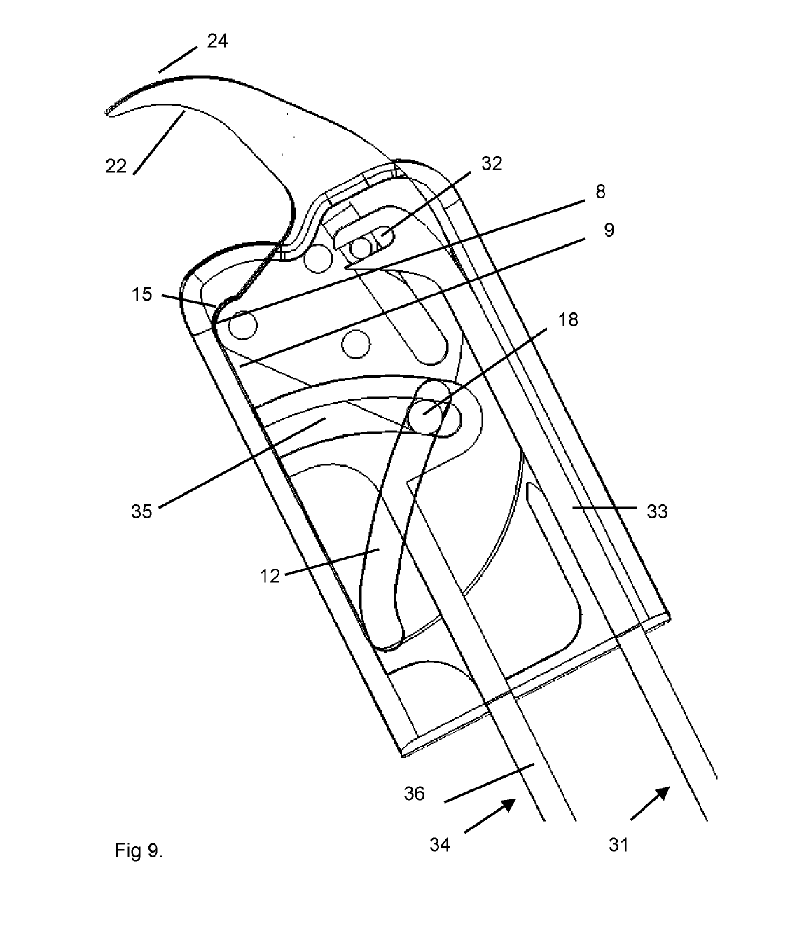

In FIG. 9 the cutting blade 23 and the cutting blade cover 13 both protrude distally from the cutting device housing 1 and cutting blade 23 is fully accommodated within the cutting blade cover 13.

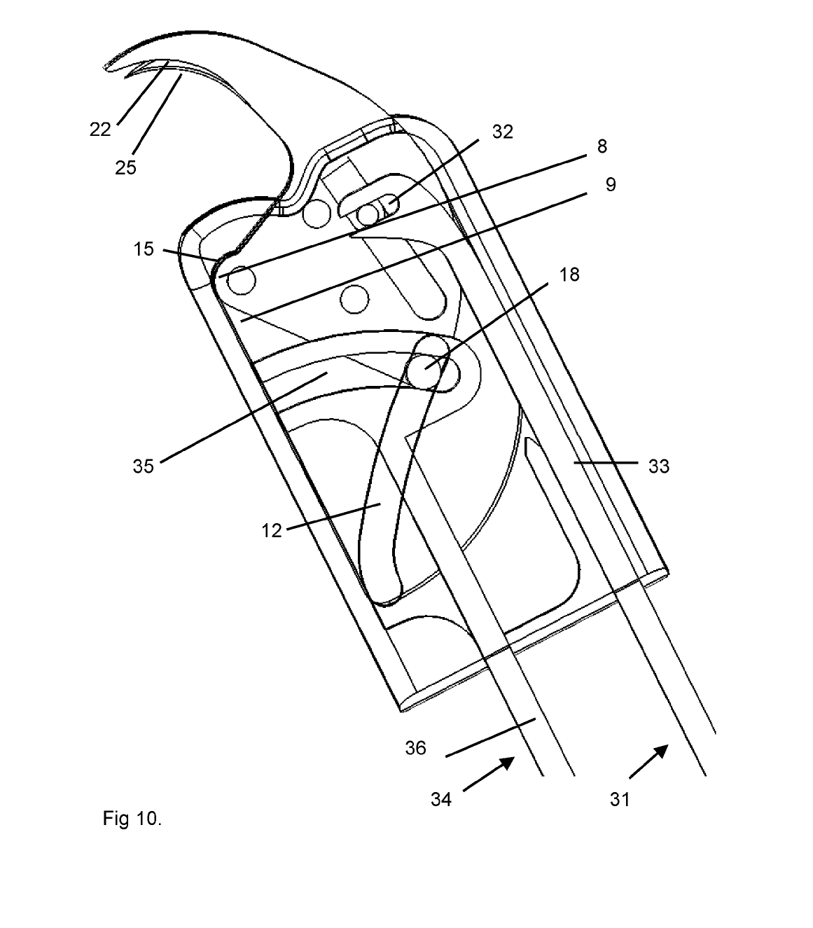

In FIG. 10 the cutting blade 23 protrudes proximally from the cutting blade cover 13.

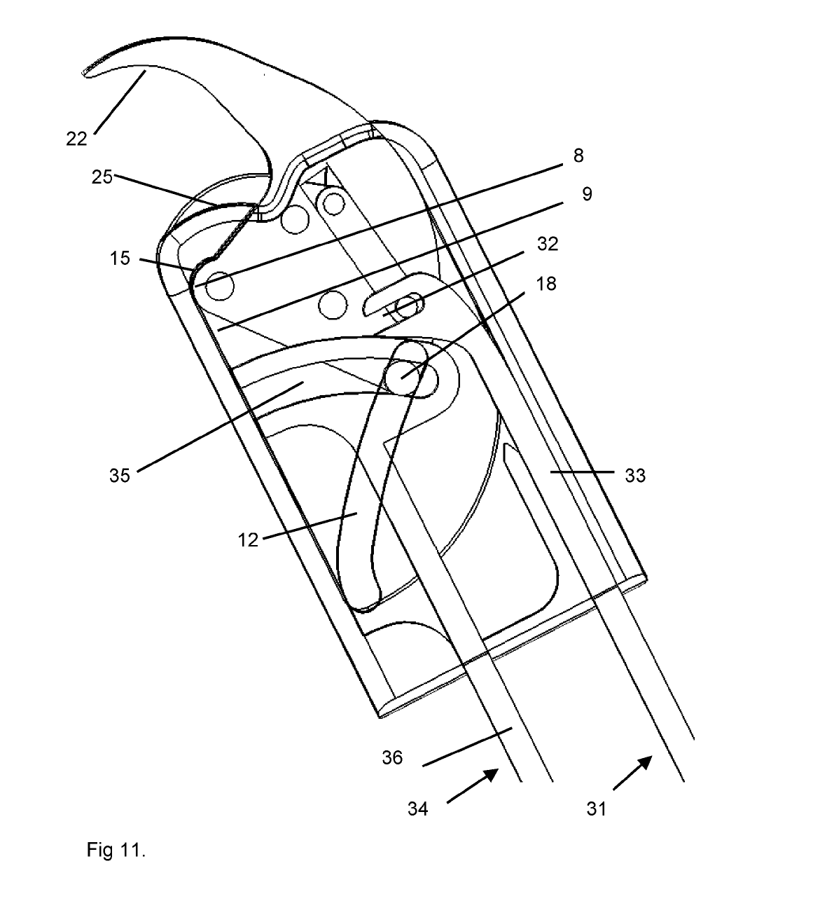

In FIG. 11 the cutting blade 23 is pushed against the edge 11 of the cutting housing 1.

The cutting assembly includes a stabbing and cutting blade 23 within a protecting cutting blade cover 13. The cutting blade 23 is moveable within the cutting blade cover 13. The cutting blade cover 13 is moveably positioned in a cutting device housing 1 or 43 (see FIGS. 2, 13, 14). The possible movement of the cutting blade 23 with respect to the cutting blade cover 13 is uniquely determined by two notches 26 and 27 on the blade 23. Notch 27 engages in a guiding groove 20 in the cover 13 and notch 26 engages in a hole 29 in a lever 28. Lever 28 in turn engages in a guiding groove 19 in the cutting blade cover 13 thus limiting the movement of notch 27 to the middle of the guiding groove 19 in the cutting blade cover 13. The guiding grooves 19 and 20 are located on respectively the front and the rear part of the cutting blade cover 13 thus allowing trajectories of the guiding grooves 19, 20 to overlap. The possible movement of the cutting blade 23 is thus uniquely determined by the contour or shape of the guiding grooves 19, 20. A cutting blade actuator 31 is engaged to the notch 30 on the lever 28 by a slot 32 and is used to move the cutting blade 23 to different positions with respect to the cutting blade cover 13 within the range of positions that is determined by the guiding grooves. A guiding groove 7 in the cutting device housing 1 prevents rotation of the cutting blade actuator 31 with respect to the cutting device housing 1 and limits the movement of the cutting blade actuator 31 to the distal and proximal direction.

The lever 28 only functions to offset the engagement of the attachment of the cutting blade actuator 31 to a more proximal position to prevent this engagement position to protrude from the cutting device housing 1 when performing the stabbing and cutting actions.

The movement of the cutting blade cover 13 within the cutting device housing 1 is limited by the shape of the cover 13 and the shape of the inner space of the cutting device housing 1.

The rounded outer side 14 of the cutting blade cover 13 is concentric with the rounded inner side 15 of the cutting blade cover 13. The width of the inner space of the cutting device housing 1 is equal to the sum of the radii of the rounded inner side 15 and the rounded outer side 14 of the cutting blade cover 13. This configuration laterally limits the movement of the cutting blade cover 13 relative to the cutting device housing 1. The movement of the cutting blade cover 13 in the distal and proximal direction within the cutting device housing 1 as well as the rotation of the cutting blade cover 13 within the cutting device housing 1 is determined by a notch 18 on the cutting blade cover 13 which can slide within a guiding groove 12 in the cutting device housing 1. Any specific position of notch 18 within the guiding groove 12 determines the position in the distal and proximal direction as well as the angle of the cutting blade cover 13 with respect to the cutting device housing 1. A cutting blade cover actuator 34 is in engagement with notch 18 via a slot 35. By moving the handle 36 of the cutting blade cover actuator 34, the cutting blade cover 13 is moved to different positions with respect to the cutting device housing 1 within the range of positions that is determined by the guiding groove 12.

A guiding groove 6 in the cutting device housing 1 prevents rotation of the cutting blade cover actuator 34 with respect to the cutting device housing 1 and limits the movement of the cutting blade cover actuator 34 to the a distal and proximal direction relative to the cutting device housing 1.

The cutting blade cover 13 of the present example incorporates three bulges 16 and an elevated ridge 17 to allow smooth movement within the cutting device housing and to create space for the actuators 31 and 34, thus compensating for any notches sticking outward of the surface of the cover and also reducing the friction of the cutting blade cover 13 within the cutting device housing 1.

The shapes of the guiding groove 12 and the slots 32 and 35 are chosen such that if the handles 33 and 36 are each moved with the same distance, the position of the cutting blade 23 does not change with respect to the cutting blade cover 13.

The combination of the position and contour of the guiding grooves 19 and 20 allow the cutting blade 23 to be positioned in a safe position within the cutting blade cover 13, protrude outwards through opening 21 of the cutting blade cover 13 for a stabbing position or to move in a cutting action through opening 22 in the cutting blade cover 13. A distal tip of the rear and the front part of the cutting blade cover 13 may be connected to each other to provide structural strength to the cutting blade cover 13. This interconnection that may be formed by a spot weld, forms an obstacle for a simple movement of the cutting blade 23 relative to the cutting blade cover 13. The notches and guiding grooves as discussed above and shown in the figures may provide a path of movement of the cutting blade 23 relative to the cutting blade cover 13 so that the distal end of the cutting blade 23 is steered around the spot weld.

Normal operation of the system: The cutting blade 23 is set in the stabbing position protruding outward of opening 21 by moving the handle 33 of the cutting blade actuator 31 in the most distal position with respect to the handle 36 of the cutting blade cover actuator 34. With the blade 23 in the stabbing position the handles of the cutting blade actuator 31 and the cutting blade cover actuator 34 are simultaneously moved in the distal direction thus pushing the cutting blade cover 13 with the protruding cutting blade 23 outward and slightly rotating until the cutting blade cover 13 has reached its maximum position. The protruding cutting blade 23 will make the initial cut through the tissue and the cutting blade cover 13 will dilate the initial cut by its wedge shape. When the cutting blade cover 13 has reached its maximum position, the cutting blade 23 will be pulled back to its safe position by moving the handle of the cutting blade actuator 31 with respect to the handle of the cutting blade cover actuator 34. The cutting device assembly is now ready to make the first cut by moving the handle of the cutting blade actuator 31 further proximally with respect to the handle of the cutting blade cover actuator 34 until the blade 23 has completed the full movement toward and onto the area 11 of the cutting device housing 1 that limits the movement of the cutting blade 23. The surgeon may now choose to rotate the whole system and make a second cutting action by moving the cutting blade 23 upwards or to first move the cutting blade 23 upwards into the safe position before rotating the system and repeating the initial cutting movement. This procedure is repeated until the full 360° has been cut and the uterus is fully resected.

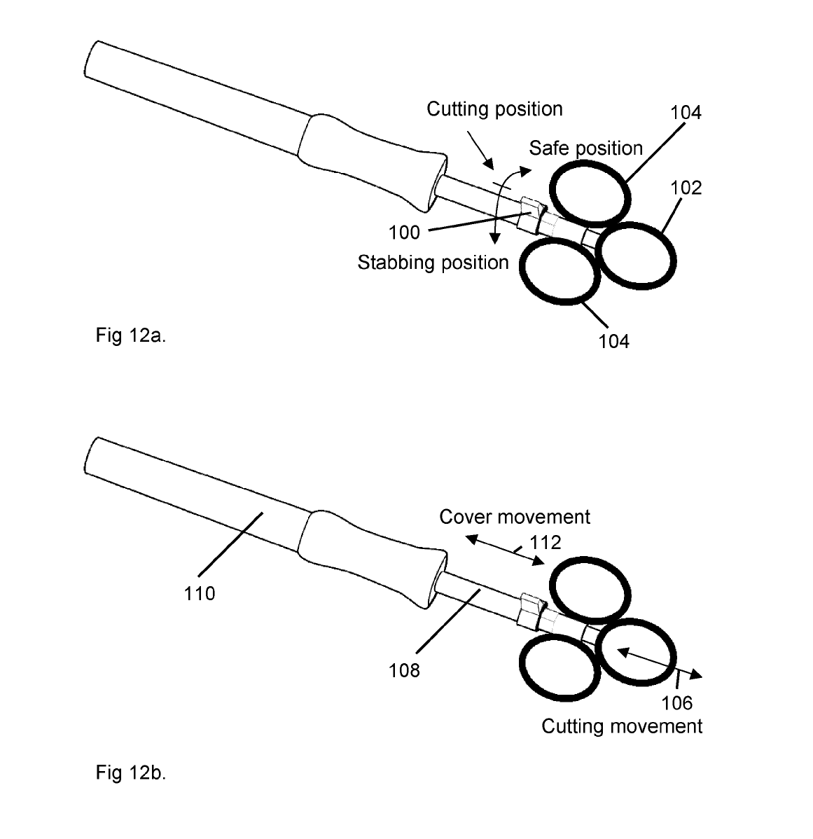

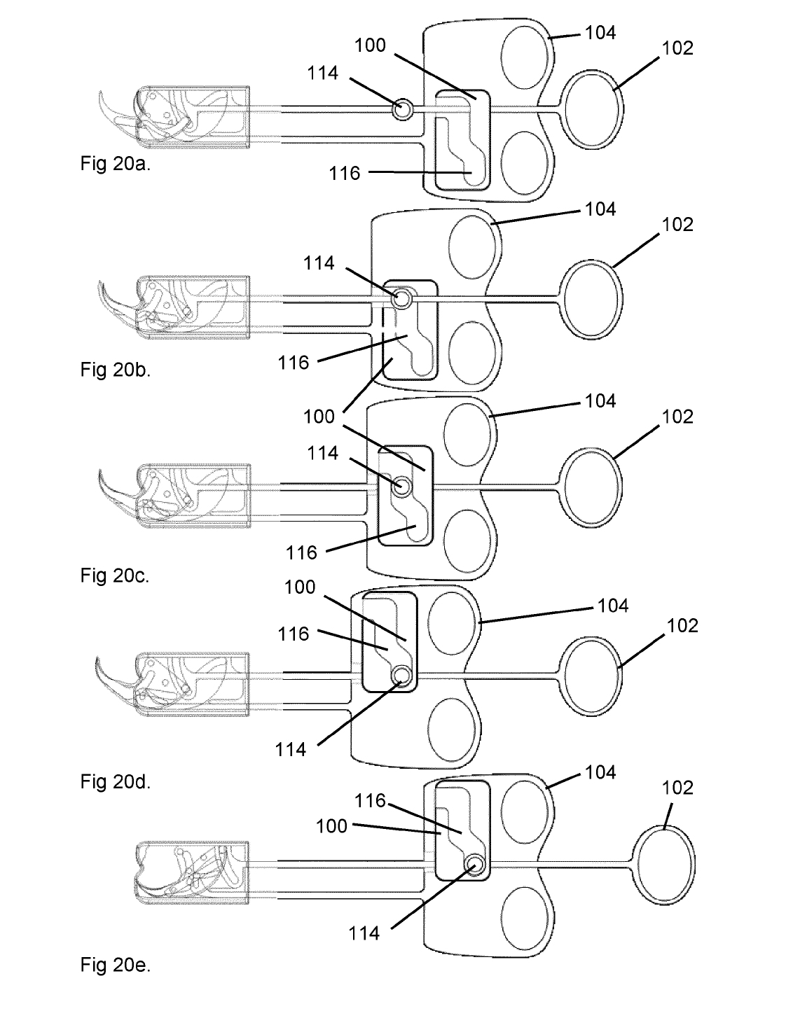

FIG. 12 shows an example of an embodiment via which the cutting blade cover actuator and the cutting blade actuator may be operated. Rotatable knob 100 may be positioned in a stabbing position in which the cutting blade 23 protrudes distally from the cutting blade cover 13 as shown in FIG. 8. Rotatable knob 100 may be positioned in a cutting position in which movement of handle 102, relative to handle 104 is possible as indicated with arrow 106. The rotatable knob 100 may also be positioned in a safe position in which the cutting blade is positioned entirely within the cutting blade cover 13. The cutting blade cover may be moved in and out of the cutting device housing 1 by movement of inner tube 108 relative to outer tube 110 as indicated by arrow 112. The cutting device housing 1 may be connected directly or indirectly to the outer tube 110. The rotatable knob 100 may also be embodied as a shiftable knob 100 as shown in the exemplary embodiment of FIGS. 20a-20e.

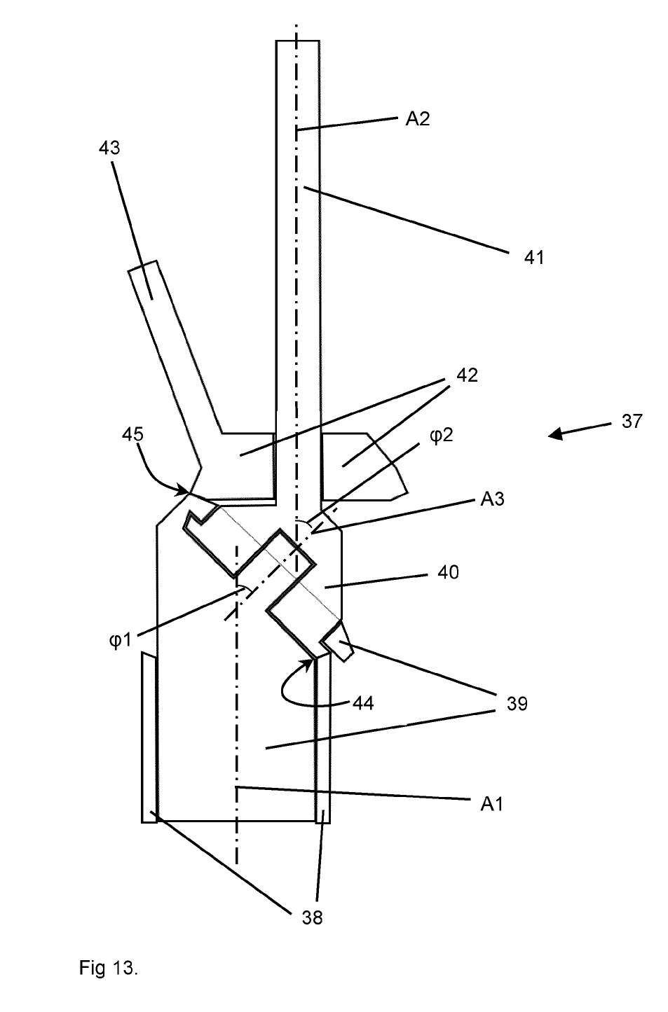

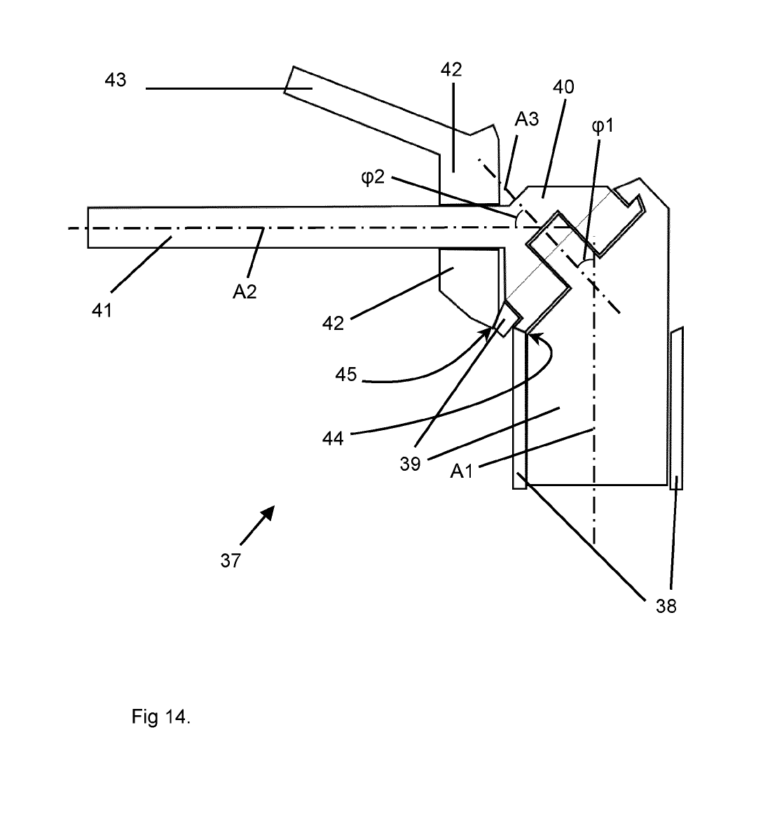

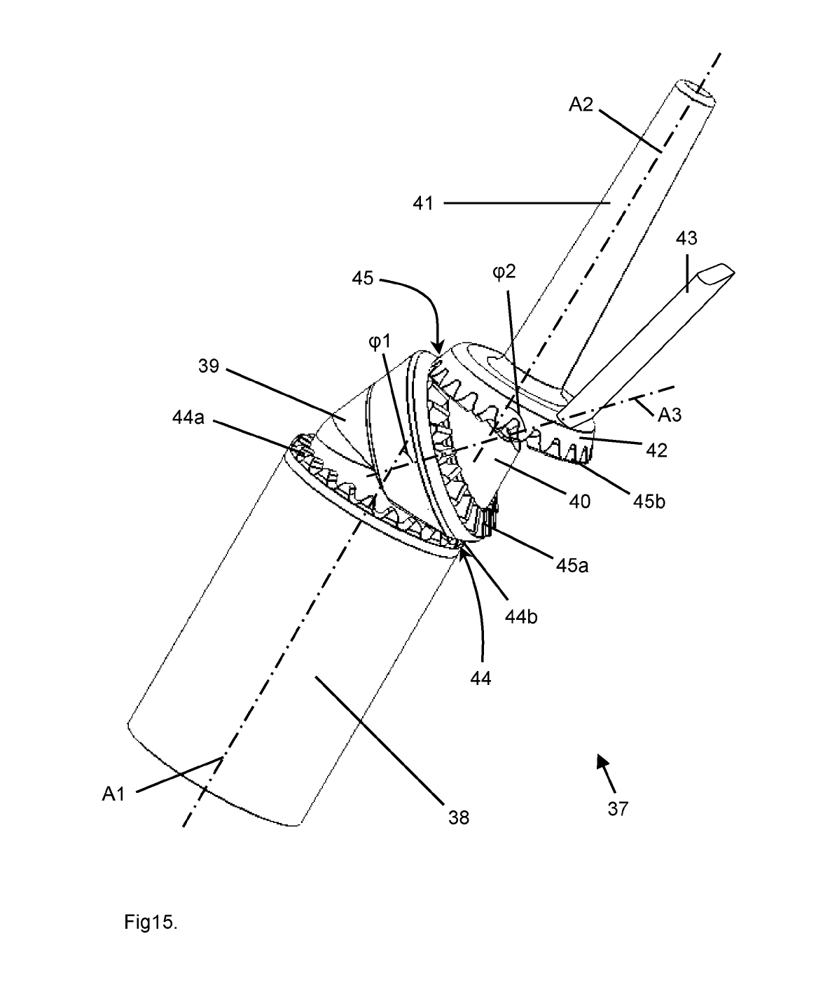

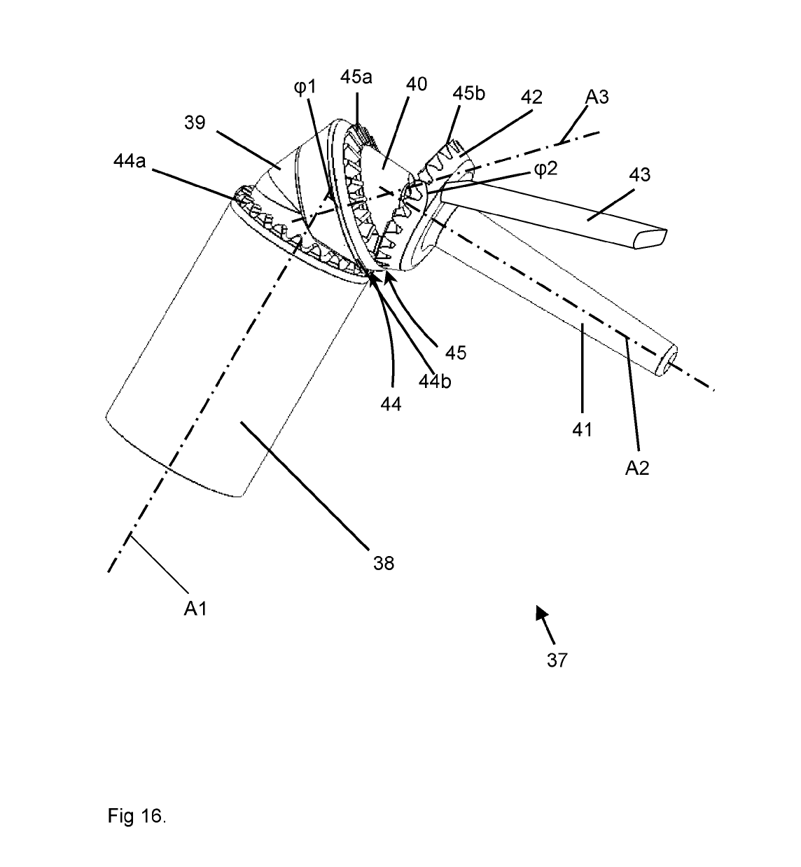

FIG. 13 shows a cross section view of a distal end of an example of an embodiment of a hystorectomy assembly in a first position and FIG. 14 shows a similar view of the example of FIG. 13 in a second position. FIG. 15 shows a perspective view of the example in of FIG. 13 in that position and FIG. 16 shows a perspective view of the example of FIG. 14 in that position.

The reference numbers of FIG. 13-16 relate to the following:
37. Mobilizer assembly.
38. Outer tube.
39. Inner tube.
40. Mobilizer element frame.
41. Mobilizer element. The mobilizer element is also named manipulator element in this application.
42. Cutting device holder.
43. Blade cutting device housing or cutting device housing.
44. Gear wheel connection between outer tube and mobilizer element frame, consisting of gear wheel 44a and gear wheel 44b
44a. Gear wheel on the outer tube 38
44b. Gear wheel on the mobiliser element frame 40
45. Gear wheel connection between inner tube and blade holder, consisting of gear wheel 45a and gear wheel 45b
45a. Gear wheel on the inner tube 39
45b. Gear wheel on the cutting device holder 42

When rotating the system, the mobiliser element 41 is automatically moved in a direction which is optimal for the cutting procedure and allows optimal vision for the surgeon.

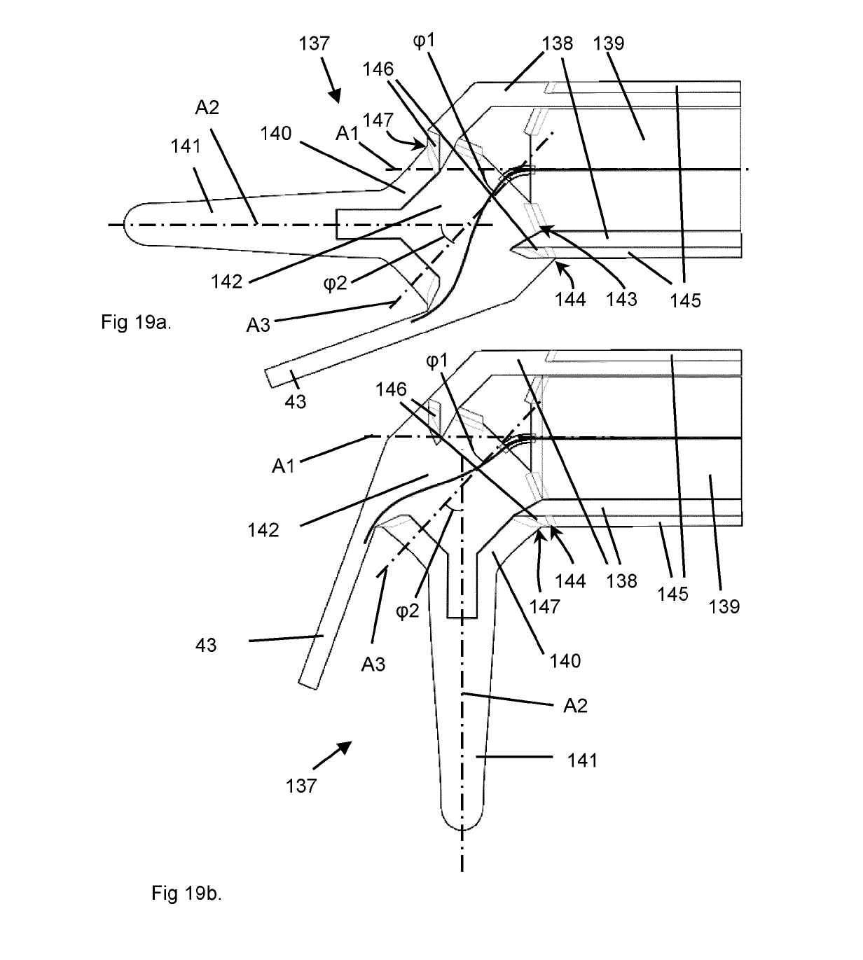

FIG. 19a shows a cross section view of a distal end of a second embodiment of a hystorectomy assembly in a first position and FIG. 19b shows a similar view of the example of FIG. 19a in a second position.

The reference numbers of FIGS. 19a and 19b relate to the following:
43. Blade cutting device housing or cutting device housing.
137. Mobilizer assembly.
138. A second tube.
139. A first tube.
141. Mobilizer element that may include a mobilizer element frame 140. The mobilizer element 141 is also named manipulator element in this application.
142. A Cutting device holder.
143. A gear wheel connection between first tube 139 and the cutting device holder 142,
144. A gear wheel connection between a third tube 145 and a rotation ring 146.
145. A third tube
146. A rotation ring
147. A gear wheel connection between the rotation ring 146 and the mobilizer element 141 or the mobilizer element frame 140.

When rotating the first tube 139 relative to the second and third tubes 138, 145, the mobiliser element 141 is automatically moved in a direction which is optimal for the cutting procedure and allows optimal vision for the surgeon.

In a first embodiment, the cutting device 43 may be embodied as a cutting device 1 as described with reference to FIGS. 1-12

In an other embodiment, the cutting device 43 could be replaced by a different dissection mechanism, like thermal dissection, ultrasone dissection or any other technique or combination of techniques that can fit to this system.

Explanation of the Mobiliser System.

The system includes a handle having an inner tube 39 and an outer tube 38 which can rotate with respect to each other, but can not move in the longitudinal direction with respect to each other.

On the upper side of the inner tube a mobilizer element frame 40 is connected such that it can rotate around an axis A3 which has an angle of φ1 with respect to axis A1 of the inner tube 39. The mobilizer element frame 40 can only rotate but it can not move in the direction of the axis of rotation with respect to the inner tube 39. The mobilizer element frame 40 is engaged with the outer tube 38 through a gearwheel 44 or any other mechanism that ensures that the rotation of the outer tube 38 with respect to the inner tube 39 is passed on to a similar rotation of the mobilizer element frame 40. The mobilizer element frame 40 contains a mobilizer element 41 with an axis that has an angle of φ2 with respect to the angle of the axis of rotation of the mobilizer element frame 40. A cutting device holder 42 is place around the mobilizer element 41 such that it can rotate around the mobilizer element 41, but it can not move in the direction of the axis of the mobilizer element 41 with respect to the mobilizer element 41. The cutting device holder 42 engages with the inner tube 39 through a gearwheel 45 or any other mechanism that ensures that the rotation of the inner tube 39 with respect to the mobilizer element holder 40 is passed on to a similar rotation of the cutting device holder 42.

A cutting device housing 1 or 43 is fixedly attached to the cutting device holder 42 at a certain angle with respect to the axis of the mobilizer element 41.

A second embodiment of the system, of which an example is shown in FIGS. 19a and 19b, includes a handle having an first tube 139, a second tube 138 and a third tube 145 which can rotate with respect to each other around an first axis A1. The respective tubes can not move in the longitudinal direction with respect to each other.

On the upper side of the second tube 138 a cutting device holder 142 is connected such that it can rotate around an axis A3 which has an angle of φ1 with respect to axis A1 of the outer tube 138. The cutting device holder 142 can only rotate but it can not move in the direction of the axis of rotation with respect to the outer tube 138. The cutting device holder 142 is engaged with the first tube 139 through a gearwheel 143 or any other mechanism that ensures that the rotation of the inner tube 139 with respect to the outer tube 138 is passed on to a similar rotation of the cutting device holder 142. A rotation ring 146 is connected such that it can rotate around axis A3, but it cannot move in the direction of the axis of rotation with respect to the cutting device holder 142. The third tube 145 is engaged with the a proximal gear of rotating ring 146 through a gearwheel 144 or any other mechanism that ensures that the rotation of third tube 145 with respect to the first tube 139 is passed on to a similar rotation of the rotation ring 146. A distal gear of the rotation ring 146 is engaged with a gear 147 that is part of the mobilizer element 141 or the mobilizer element frame 140 of the mobilizer element 141. Any other mechanism that ensures that the rotation of the rotation ring 146 relative to the cutting device holder 142 is passed on to a similar rotation of the mobilizer element 141 or the mobilizer element frame 140. The axis A2 of the mobilizer element 141 makes an angle of φ2 with the axis of rotation of the cutting device holder 142. A mobilizer element 141 or the mobilizer element frame 140 is placed on the cutting device holder 142 such that it can rotate around its axis A2 on the cutting device holder 142, but it can not move in the direction of the axis A2 relative to the cutting device holder 142. The mobilizer element 141 or mobilizer element frame 140 engages the third tube 145 through the gearwheel 147, the rotation ring 146 and the gearwheel 144 or any other mechanism that ensures that the rotation of the third tube 145 with respect to the first tube 139 is passed on to a similar rotation of the mobilizer element 141.

A cutting device housing 1 or 43 is fixedly attached to the cutting device holder 142 at a certain angle with respect to the axis of the mobilizer element 141.

Normal functioning of the system: When the outer tube 38 is held in a stable position and the inner tube 39 is rotated with respect to the outer tube 38, the mobilizer element 41 moves in a cone shape, but the mobilizer element 41 does not rotate around its longitudinal axis with respect to the outer tube 38 because of the gear wheel engagement between the outer tube 38 and the mobilizer element frame 40. The person operating the system can thus move the mobilizer element to any position on the cone shape by holding the outer tube 38 and rotating the inner tube 39 with respect to the outer tube 38 without the risk that the mobilizer element 41 will either dislocate from the uterus by rotating counter clockwise (and thus unscrewing from the uterus) or the risk that the mobilizer element 41 will unintentionally screw itself further into the uterus with the risk of protruding into the abdominal cavity.

When the inner tube 39 is rotated with respect to the outer tube 38 the cutting device holder 42 rotates around the mobilizer element 41.

In this embodiment, when both φ1 and φ2 are 45°, the axis of the mobilizer element 41 moves from parallel to the axis of the outer tube 38 (FIG. 12) to perpendicular to the axis of the outer tube 38 (FIG. 13). The parallel position corresponds to positioning the uterus in a position whereby it is stretched towards the intestines, revealing the side of the uterus towards the bladder. The 90° angle pushes the uterus to a forward lying position towards the bladder, revealing the side of the uterus towards the intestines. An indicator on the outer tube 38 can show in which position the outer tube should be held to achieve these intended angles.

In other embodiments the angles can be varied to allow for different shapes and axis of the cone movement of the mobilizer element 41.

In the second embodiment the third tube 145 can rotate around the second tube 138 or it can be rotationally fixed to the second tube 138. When the second tube 138 and the third tube 145 are held in a stable position and the first tube 139 is rotated with respect to the second tube 138, the axis A2 of the mobilizer element 141 moves in a cone shape, but the mobilizer element 141 does not rotate around its longitudinal axis with respect to the second tube 138 and the third tube 145 because of the gear wheel engagement between the third tube 145, the rotation ring 146 and the mobilizer element 141 or the mobilizer element frame 140. The person operating the system can thus move the mobilizer element 141 to any position on the cone shape by rotationally fixing the third tube 145 to the second tube 138, holding the third tube 145 and rotating the first tube 139 with respect to the third tube 145 without the risk that the mobilizer element 141 will either dislocate from the uterus by rotating counter clockwise (and thus unscrewing from the uterus) or the risk that the mobilizer element 141 will unintentionally screw itself further into the uterus with the risk of protruding into the abdominal cavity.

When the third tube 145 is rotationally fixed to the second tube 138 and the first tube 139 is rotated with respect to the third tube 145 the cutting device holder 142 rotates around the mobilizer element 141.

In this embodiment, when both φ1 and φ2 are 45°, the axis A2 of the mobilizer element 141 moves from parallel to the axis A1 of the second tube 138 (FIG. 20a) to perpendicular to the axis A1 of the second tube 138 (FIG. 20b). The parallel position corresponds to positioning the uterus in a position whereby it is stretched towards the intestines, revealing the side of the uterus towards the bladder. The 90° angle pushes the uterus to a forward lying position towards the bladder, revealing the side of the uterus towards the intestines. An indicator on the second tube 138 can show in which position the second tube 138 should be held to achieve these intended angles.

In other embodiments the angles can be varied to allow for different shapes and axis of the cone movement of the mobilizer element 141.

When the first tube 139 is not rotated with respect to the second tube 138 and the third tube 145 is rotated with respect to the second tube 138, the mobiliser element 141 will rotate around its own axis A2, without changing the direction of the axis A2, and without changing the position of the cone in the cone shape. That can be done when the mobilizer element 141 has to be screwed into the uterus.

Normal operation of the system: The outer tube 38 or 138 is held in a position according to the indicator and the inner tube 39 or 139 is rotated until the axis A2 of the mobilizer element 41 or 141 is parallel to the axis A1 of the outer tube 38 or 138. The system is then inserted into the vagina and in the first embodiment the mobilizer element 41 is screwed into the uterus by rotating the whole system, without allowing any rotation of the inner tube 39 with respect to the outer tube 38, or by any other mechanism suited to attach the mobilising element 41 to the uterus. In the second embodiment the mobiliser element 141 is screwed into the uterus by rotating the third tube 145 with respect to the second tube 138 without allowing any rotation of the first tube 139 or the second tube 138, The end position of the system should correspond with the optimal rotational position as revealed by the indicator. The system is now ready to be used as a manipulator, whereby the angle of the mobilizer element 41 or 141 is manipulated by rotating the inner tube 39 or the first tube 139 and holding the outer tube 38 or the second tube 138 in its optimal position as revealed by the indicator. In the second embodiment the third tube 145 should then be rotationally fixed relative to the second tube 138, allowing a similar operation as in the first embodiment.

When the final resection has to be performed and the surgeon elects to perform the final resection with the stabbing cutting mechanism of the cutting device 1, the cutting device housing 1, 43 is moved to the optimal position for the initial cut using the stabbing mechanism. In order to aid the surgeon in selecting the optimal position, the top of the blade cutting device housing may contain a light source, which light can be seen from the abdominal side. The light allows the surgeon to double check that no major arteries or veins are still present in the cutting region. By rotating the inner tube 39 with respect to the outer tune 38, the cutting device housing 43 is set in the optimal rotational position. The mobilizer element 41 automatically moves the uterus to a position whereby the tissue to be cut is stretched a little, the uterus is moved away from organs to avoid damage by the cutting action and the surgeon has optimal vision of the cutting site. The surgeon can now perform the stabbing action followed by the first cutting action. After the first cutting action the position of the blade cutting device housing 43 is rotated by rotating the inner tube 39 with respect to the outer tube 38. This automatically moves the mobilizer element 41 to a new optimal position. The cutting action is again performed and this procedure is repeated until the full 360° has been cut and the final resection completed. The cutting blade 23 is set in the safe position and the cutting blade cover 13 together with the cutting blade 23 in safe position are moved back into the cutting device housing 1 by simultaneously pulling both handles of the cutting blade actuator 31 and cutting blade cover actuator 34.

The activation of the various actions are performed by distinct movements of the handles 102 and 104. The movement of the cutting blade 23 from a safe position to a stabbing position is done by rotating the knob 100 to the stabbing position (see FIG. 11). The rotation of the knob 100 allows a movement of the two handles 102, 104 with respect to outer tube 110 and prevents any other movement of the handles 102, 104 with respect to each other. In the stabbing positions the handles 102, 104 can be moved towards the outer tube 110 to perform the stabbing action. When the knob 100 is set in the safe position, this results in freezing the position of the handles 102, 104 with respect to each other with the blade in the safe position. Also in the safe position the handles 102, 104 can be moved together with respect to the outer tube 110 in order to withdraw the cover 13 or push it outwards through an existing hole in the fornix. When the knob 100 is set in the cutting position, it enables movement of the handles 102, 104 with respect to each other moving the blade 23 from the safe position to a fully cut position and vice versa. This position of the knob 100 prevents movement of the handle 104 operating the cutting blade housing with respect to outer tube 110.

FIG. 20a-20c show an example of an embodiment of an operating assembly of the cutting device described above. The embodiment of the operating assembly of the cutting device may include a knob 100 that is slidably or rotatably connected with a part that includes the second handle 104. The knob 100 may comprise a slot 116 that cooperates with a notch 114 on the first handle 102 or a part that is fixedly connected with the first handle 102. The knob 100 may have three positions, i.e.:
a first end position (shown in FIGS. 20a and 20b) in which the first and the second handle 102, 104 are moveable relative to each other and in which the cutting blade 23 protrudes proximally from the cutting blade cover 13 and can make a cutting movement by moving the second handle 104 relative to the first handle 102;
an intermediate position (shown in FIG. 20c) in which the first and the second handle 102, 104 are fixed relative to each other and in which the cutting blade 23 is completely covered by the cutting blade cover 13; and
a second end position (shown in FIGS. 20d and 20e) in which the first and the second handle 102, 104 are fixed relative to each other and in which the cutting blade 23 is protruding distally from the cutting blade cover 13. In the second end position both the first and the second handle 102, 104 may be move relative to the cutting device housing 1 to withdraw the cutting blade 23 with the cutting blade cover 13 into the cutting device housing 1 or to push the cutting blade 23 with the cutting blade cover 13 out of the cutting device housing 1 to protrude from that cutting device housing 1. By that movement the cutting blade 23 may stab through the fornix of the uterus.

Although illustrative embodiments of the present invention have been described above, in part with reference to the accompanying drawings, it is to be understood that the invention is not limited to these embodiments. The cutting device may also be used in other applications, for example removel of a part of the intestine.

It should be noted that another invention is contemplated that is directed to a manipulator assembly without a cutting device. Such a manipulator assembly can be embodied as the hysterectomy assembly described in claims 11-29 but without the features that relate to the cutting device. Such a manipulator assembly may be the object of a divisional application. More particularly, the handle with an inner part and an outer tube that are rotatable relative to each other around a first axis A1 and a manipulator element that is connected with the handle so as to moveable along a cone without rotating around its own, second axis A2 may be beneficial relative to the known manipulators. Especially the gear assembly of the disclosed hysterectomy assembly may be used in such a manipulator assembly.

Variations to the disclosed embodiments can be understood and effected by those skilled in the art in practicing the claimed invention, from a study of the drawings, the disclosure, and the appended claims. Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, it is noted that particular features, structures, or characteristics of one or more embodiments may be combined in any suitable manner to form new, not explicitly described embodiments.

1. A cutting device including:
a cutting device housing (1, 43);
a cutting blade cover (13) moveably arranged in the cutting device housing (1, 43);
a cutting blade (23) moveably arranged in the cutting blade cover (13);
a cutting blade actuator (31) for moving the cutting blade (23) relative to the cutting blade cover (13); and
a cutting blade cover actuator (34) for moving the cutting blade cover (13) relative to the cutting device housing (1, 43),
wherein the cutting blade (23) may be positioned relative to the cutting blade cover (13):
to protrude distally from the cutting blade cover (13);
to protrude proximally from the cutting blade cover (13); and
to be completely accommodated within the cutting blade cover (13).

2. The cutting device of claim 1, wherein the cutting blade cover (13) may be positioned with the cutting blade cover actuator (34) relative to the cutting device housing (1):
to be fully accommodated within the cutting device housing (1) together with the cutting blade (23); and
to protrude distally from the cutting device housing (1) together with the cutting blade (23).

3. The cutting device of claim 1 or 2, wherein the cutting device housing (1) includes a substantially plate-like shaped front part and a rear part between which the cutting blade cover (13), the cutting blade (23) as well as distal parts of the cutting blade actuator (31) and cutting blade cover actuator (34) are moveably arranged.

4. The cutting device according to claim 3, wherein the cutting device housing (1) includes:
an opening (2) configured to allow protrusion of the cutting blade cover (13) and the cutting blade (23);
an opening (3) configured to allow passage of the cutting blade cover actuator (34);
an opening (4) configured to allow passage of the cutting blade actuator (31);
a guiding groove (6) for the cutting blade cover actuator (34);
a guiding groove (7) for the cutting blade actuator (31);
a partition wall (5) forming one side of a guiding groove (6) for the cutting blade cover actuator (34) and one side of a guiding groove (7) for the cutting blade actuator (31);
a rounded corner (8) serving as a hinge for the cutting blade cover (13);
two sides (9, 10) of the space in the cutting device housing (1) bounding a guiding groove for the cutting blade cover (13);
an area (11) of the cutting device housing (1) towards which the cutting blade (23) cuts; and/or
a guiding groove (12) for a notch (18) of the cutting blade cover (13).

5. The cutting device according to claim 1, wherein the cutting blade cover (13) includes:
a front part and a rear part between which the cutting blade (23) is accommodated
a rounded outer edge (14);
a rounded inner edge (15);
elevated bulges (16) reducing friction of the sliding movement of the cutting blade cover (13) within the cutting device housing (1).
an elevated ridge (17) reducing friction of the sliding movement of the cutting blade cover (13) within the cutting device housing 1.
a notch (18) that moves within guiding groove (12) of the cutting device housing (1) and together with rounded outer and inner edge (14, 15) limits the freedom of movement of the cutting blade cover (13) within the cutting device housing (1) to one path and related angle, the notch (18) also being engaged by the cutting blade cover actuator (34);
a guiding groove (19) in a front part of the cutting blade cover (13) for co-operation with a notch (30) of a lever (28) of the cutting blade (23).
a guiding groove (20) in the rear part of the cutting blade cover (13) for co-operation with one of the notches (27) of the cutting blade (23).
an opening (21) in the cutting blade cover (13) allowing protrusion of the cutting blade (23) for the stabbing function; and/or
an opening (22) in the cutting blade cover (13) allowing for protrusion of the cutting blade 23 for the cutting function.

6. The cutting device according to claim 1, wherein the cutting blade (23) includes:
a sharp edge (24) for the stabbing function;
a sharp edge (25) for the cutting function; and/or
a notch (27) that moves within a guiding groove (20) of the cutting blade cover (13).

7. The cutting device according to claim 6, including:
a lever (28) having a hole (29) and a notch (30);
a notch (26) that is part of the cutting blade (28) and that fits in the hole (29) of the lever (28);
wherein the notch (30) of the lever (28) engages the guiding groove (20) of the cutting blade cover (13) and is engaged by the cutting blade actuator (31).

8. The cutting device according to claim 7, wherein the cutting blade actuator (31) includes:
a slot (32) for engaging the notch (30) of the lever (28); and
a handle (33) that slides in the guiding groove (7) of the cutting device housing (1).

9. The cutting device according to claim 5, wherein the cutting blade cover actuator (34) includes:
a slot (35) for the notch (18) of the cutting blade cover (13); and
a handle (36) that slides in the guiding groove (6) of the cutting device housing (1) for the cutting blade cover actuator (34).

10. (canceled)

11. A hysterectomy assembly including
a handle (38, 39);
a manipulator or mobilizer element (41, 141) that is positionable at various angles relative to the handle (38, 39);
a cutting device (1; 43) that is mounted on a cutting device holder (42; 142);
wherein the hysterectomy assembly (37; 137) is configured to automatically move the manipulator or mobilizer element (41; 141) and the cutting device (1; 43) relative to each other so that the uterus is pushed to a direction opposite of a cutting position where the cutting device (1; 43) is performing a cutting operation, thereby creating as much space and visibility of the cutting position.

12. The hysterectomy assembly according claim 11, wherein the handle (38, 39) includes in inner tube (39) and an outer tube (38) both extending along a first axis (A1).

13. The hysterectomy assembly according to claim 12, wherein the mobilizer element (41) extends along a second axis (A2) and is fixedly connected to a mobilizer element frame (40), the mobilizer element frame (40) being rotatably connected with the inner tube (39) of the handle (38, 39) and rotatable around a third axis (A3) that includes a first angle (φ1) with the first axis (A1) and that includes a second angle (φ2) with the second axis (A2).

14. The hysterectomy assembly according to claim 13, wherein the cutting device holder (42) is rotatably mounted on the mobilizer element (41) or the mobilizer element frame (40) around a second axis (A2).

15. The hysterectomy assembly according to claim 14, wherein a distal end of the outer tube (38) includes a gear that cooperates with a gear on the mobilizer element frame (40) to form a gear wheel connection (44), so that a rotation of the inner tube (39) relative to the outer tube (38) results in a movement of mobilizer element (41) along a cone surface without rotating around its own axis (A2) with respect to the outer tube (38).

16. The hysterectomy assembly according to claim 15, wherein a distal end of the inner tube includes a gear that cooperates with a gear on the cutting device holder (42) to form a gear wheel connection (45) between the inner part (39) and the cutting device holder (42), so that a rotation of the inner part (39) relative to the outer tube (38) results in a corresponding rotation of the cutting device holder (42) around the mobilizer element (41).

17. The hysterectomy assembly according to claim 11, wherein the handle (138,139) includes in first tube (139) and an second tube (138) both extending along a first axis (A1), the first tube (139) being rotatably accommodated within the second tube (138) around the first axis (A1).

18. The hysterectomy assembly according to claim 17, wherein the cutting device holder (142) is connected with the second tube (138) of the handle (138, 139) to be rotatable around a third axis (A3) that includes a first angle (φ1) with the first axis (A1),

19. The hysterectomy assembly according to claim 18, wherein the cutting device holder (142) includes a gear (143), wherein a distal end of the first tube (139) includes a gear (143b) that engages the gear (143) of the cutting device holder (142) so that a rotation of the first tube (139) relative to the second tube (138) results in a rotation of the cutting device holder (142) around the third axis (A3).

20. The hysterectomy assembly according to 19, wherein the mobilizer element (141) extends along a second axis (A2) and is connected with the cutting device holder (142), the second axis (A2) including a second angle (φ2) with the third axis (A3).

21. The hysterectomy assembly according to claim 20, wherein the mobilizer element (141) is mounted on the cutting device holder (142) so as to be rotatable around the second axis (A2).

22. The hysterectomy assembly according to claim 21, including:
a third tube (145) in which the second tube (138) is accommodated, wherein the third tube (145) in a first condition is rotationally fixed relative to the second tube (138) and in a second condition is rotatable relative the second tube (138) around the first axis (A1)
a gear (144) formed on a distal end of the third tube (145);
a gear ring (146) including distal and proximal gear toothing that is mounted on the distal end of the second tube (138) so as to be rotatable around the third axis (A3) and the proximal gear toothing being engaged by the gear (144) of the third tube (145);
a gear (147) that is part of the mobilizer element (141) and that is engaged by the distal gear toothing of the gear ring (146).

23. The hysterectomy assembly according to claim 22, wherein in the first condition a rotation of the first tube (139) relative to the second tube (138) results in a movement of mobilizer element (141) along a cone surface without rotating around its own axis (A2) with respect to the second tube (138).

24. The hysterectomy assembly according to claim 22, wherein in the second condition, rotation of the third tube (145) relative to the second tube (138) and the first tube (139) result in a rotation of the mobilizer element (141) around its own second axis (A2) without moving along a cone and without rotation of the cutting device holder (142) along the third axis (A3).

25. The hysterectomy assembly according to claim 13, wherein the first angle (φ1) is in the range of 30°-60°

26. (canceled)

27. (canceled)

28. The hysterectomy assembly according to claim 11, including an operation assembly that comprises:
a first handle (102) that is connected one of the handles (33, 36) of the cutting blade actuator (31) and cutting blade cover actuator (34);
a second handle (104) that is connected to the other one of the handles (33, 36) of the cutting blade actuator (31) and cutting blade cover actuator (34).

29. The hysterectomy assembly according to claim 28, wherein the operation assembly includes:
a knob (100) that is slidably or rotatably connected with a part that includes the second handle (104), the knob (100) comprising a slot (116) that cooperates with a notch (114) on the first handle (102) or a part that is fixedly connected with the first handle (102), wherein the knob (100) that has three positions, i.e.:
a first end position in which the first and the second handle (102, 104) are moveable relative to each other and in which the cutting blade (23) protrudes proximally from the cutting blade cover (13) and can make a cutting movement by moving the second handle (104) relative to the first handle (102);
an intermediate position in which the first and the second handle (102, 104) are fixed relative to each other and in which the cutting blade (23) is completely covered by the cutting blade cover (13); and
a second end position in which the first and the second handle (102, 104) are fixed relative to each other and in which the cutting blade (23) is protruding distally from the cutting blade cover (13).

30. (canceled)
